(19) United States
(12) Patent Application Publication
(10) Pub. No.: US 2026/0165565 A1
(43) Pub. Date: Jun. 18, 2026

(54) BORESCOPE ARTICULATION CONTROL MECHANISMS AND RELATED SYSTEMS AND METHODS

(71) Applicant: Xenocor, Inc., Salt Lake CIty, UT (US)

(72) Inventors: David Thomas Van Ness, Salt Lake City, UT (US); Brandon Michael Kessler, Taylorsville, UT (US); Jonathan Allen Bruns, Salt Lake City, UT (US); Michael James Bills, Salt Lake City, UT (US)

(21) Appl. No.: 19/418,636

(22) Filed: Dec. 12, 2025

Related U.S. Application Data
(60) Provisional application No. 63/733,416, filed on Dec. 12, 2024.

(51) Int. Cl. A61B 1/005

(52) U.S. Cl. CPC A61B 1/0057

(57) ABSTRACT

Borescopes, such as laparoscopes and endoscopes, configured with articulation control mechanisms and/or automatic braking/locking features. In some preferred embodiments, a borescope may comprise an articulation control mechanism comprising an articulation control piece, such as a spherical or partially spherical element which may protrude from a surface of the handle of the scope. The articulation control piece may be pivotable and/or rotatable to cause a corresponding bending/articulation of a portion of a shaft of the scope, such as the tip. Preferably, the articulation control piece further comprises an automatic braking system configured to automatically re-seat the control piece once a user releases it to lock the articulable portion of the scope in its current orientation.

### CROSS-REFERENCE TO RELATED APPLICATIONS

This application claims the benefit under 35 U.S.C. § 119(e) of U.S. Provisional Patent Application No. 63/733,416, which was filed Dec. 12, 2024 and titled “BORESCOPE ARTICULATION CONTROL MECHANISMS AND RELATED SYSTEMS AND METHODS,” which is hereby incorporated herein by reference in its entirety.

### SUMMARY

Embodiments of apparatus and methods are disclosed herein that relate, at least in preferred embodiments, to borescopes and other related medical borescopes, such as laparoscopy, endoscopy, and the like. In some embodiments disclosed herein, a laparoscope or other medical borescope may be configured with articulation control mechanisms and/or automatic braking/locking features.

In some preferred embodiments, a borescope may comprise an articulation control mechanism comprising an articulation control piece, such as a spherical or partially spherical element, which may protrude from an opening in the handle of the borescope. The articulation control piece may be pivotable and/or rotatable to cause a corresponding bending/articulation of a portion of a shaft of the scope, such as an articulable tip.

The articulation control piece may further comprise an automatic braking system configured to automatically re-seat the articulation control piece once a user releases it to lock the articulable portion of the scope in its current orientation. For example, in some embodiments, a resilient element, such as a spring feature, may be used to provide a force biasing the articulation control piece towards a seated and/or locked configuration. Thus, a user may unseat and/or unlock the articulation control piece by depressing and/or otherwise applying a downward and/or inward force on the articulation control piece. This may cause the articulation control piece to unseat and/or unlock.

After this unseating/unlocking has taken place, a user may be able to pivot and/or rotate the articulation control piece to control the articulation of an articulable portion of the scope, such as a bendable tip of the scope. Once the articulable portion of the scope has been oriented in the desired direction, a user may simply release the articulation control piece. Without this force, the resilient element and/or spring feature causes the articulation control piece to reseat and/or lock in place. Because the articulation control piece is operably linked to the articulable tip or other articulable portion of the scope, this portion of the scope then also locks in the current configuration/orientation.

In some embodiments, the unseating/unlocking may be configured to be non-binary. In some such cases, the level of braking applied may be controllable. For example, the user may, in some cases, apply more downward force on the articulation control piece to selectively decrease the level of braking or, conversely, may apply less downward force on the articulation control piece to selectively increase the level of braking, depending upon the desired result. This modulation of the braking force allows the user to adjust the sensitivity of the control mechanism, thereby directly affecting the sensitivity of the steerable portion of the device as well.

In some embodiments, the articulation control piece may comprise a spherical or partially spherical element, such as a hemispherical piece. This piece may be configured to pivot in four directions—i.e., forward, backward, left, and right, which may result in bending of the articulable portion of the shaft in a corresponding direction, such as upward, downward, left, and right, respectively. Of course, some users may prefer another configuration, such as pivoting the control piece forward resulting in downward movement of the tip and pivoting backward resulting in upward movement of the tip, for example. This alternative configuration may be achieved by coupling articulation cables linked to the articulation control piece at other locations on a bendable portion of the shaft and/or tip.

In some embodiments, the articulation control piece may be pivotable and/or rotatable in more than only four directions. For example, some embodiments may allow for rotation in any desired direction within a spherical workspace or portion of a spherical workspace, which may allow for finer control of the bending/articulation of the tip or other articulable portion of the scope.

Some embodiments may further comprise additional elements to facilitate the automatic braking. For example, frictional materials may be added to the upper surface of the articulation control piece and/or to a portion of the engagement surface of the borescope that is configured to engage this upper surface in order to provide a more robust braking/locking that resists slippage.

In some embodiments, frictional material may be added to one or more portions of the assembly, such as along one or more portions of the interface between the hemispherical seat and/or another portion of the control frame and the swashplate, for example, or between one or more portions of the swashplate and the ball bearing, or between the ball bearing and one or more portions of the control frame, which may provide a similar frictional braking interface. In another embodiment, this frictional interface could be made more frictional or less frictional by way of an adjustable feature accessible by the user, whereby the user can increase or decrease the braking force as desired for her comfort or preference.

In a specific example of a medical borescope according to some embodiments, the borescope may comprise a handle. The handle may comprise an opening. A shaft may extend from the handle to a tip positioned at a distal end of the shaft. The tip may comprise an articulable tip configured to be selectively deflected or bent by a user during operation. The borescope may further comprise an articulation control mechanism, which may at least partially protrude from the opening formed in the handle in some embodiments. The articulation control piece may be operably coupled with the tip such that movement of the articulation control piece results in corresponding bending/deflection of the tip. In some embodiments, the borescope may further comprise an automatic braking system configured to automatically lock the tip in its current orientation upon releasing the articulation control piece.

In some embodiments, the articulation control piece may comprise an at least partially spherical element, such as a hemispherical or at least substantially hemispherical element in some cases. In some embodiments, the articulation control piece may be configured to unlock upon depressing the articulation control piece, either fully or, alternatively, partially to allow the user to apply as much breaking as desired within a range. Thus, non-binary braking modulation may be provided in some embodiments. Full releasing of the articulation control piece may result in full locking of the articulable tip in its current orientation, whether than be in a straight or bent/deflected orientation.

In some embodiments, the automatic braking system may comprise a biasing element configured to bias the articulation control piece towards a locked configuration in which the tip is locked in place in its current angular orientation, such as a spring.

In some embodiments, the automatic braking system may comprise one or more frictional braking surfaces. For example, in some embodiments, one or more frictional braking surface may be formed along a portion of the handle, such as adjacent to the opening, which may be configured to contact one or more corresponding braking surfaces of the articulation control piece, such as one or more frictional inserts formed on an outer surface of the articulation control piece. The frictional insert may be configured to contact the frictional braking surface upon release of the articulation control piece. In some embodiments, the frictional braking surface may comprise a ring, such as a ring extending about and/or adjacent to the opening to contact a portion of the upper surface of the articulation control piece upon release thereof.

In another specific example of a medical borescope according to some embodiments, the borescope may comprise a handle comprising an articulation control piece. An articulable tip may be coupled with the handle, such as positioned at a distal end of a shaft extending from the handle. The articulable tip may be operably coupled with the articulation control piece to allow a user to pivot the articulable tip in one or more directions, or in some cases in at least four directions, using corresponding movements of the articulation control piece. The articulation control piece may be configured to unlock upon depressing the articulation control piece and automatically lock upon releasing the articulation control piece.

In some embodiments, the articulation control piece may be biased towards a seated position in which a frictional interface frictionally locks an angular orientation of the articulable tip.

In some embodiments, the frictional interface may be provided by a frictional insert or another similar frictional surface formed along at least a portion of an outer surface of the articulation control piece.

In some embodiments, the frictional interface may be provided by a frictional braking surface formed along a portion of an inner surface of the handle adjacent to an opening through which the articulation control piece at least partially extends. In some cases, this braking surface may be formed, at least in part, by a ring, such as an O-ring.

In some embodiments, the articulation control piece may comprise a spherical or an at least partially spherical element.

In some embodiments, the articulation control piece may be configured such that partial depression of the articulation control piece selectively reduces a braking force on the articulation control piece to allow for non-binary braking modulation. Preferably, a complete release of the articulation control piece results in full application of the braking force to lock the articulable tip in place in its current orientation.

In an example of a method for controlling articulation of a tip of a medical borescope according to some implementations, the method may comprise depressing an articulation control piece to unlock an articulable tip of the medical borescope. In some implementations, the articulation control piece may comprise an at least partially spherical piece and/or may protrude from an opening in an upper surface of a handle of the borescope. The method may further comprise pivoting the articulation control piece in a selected direction to deflect the articulable tip in a corresponding direction and releasing the articulation control piece to automatically lock the articulable tip in its current angular orientation.

Some implementations may further comprise partially depressing the articulation control piece to selectively reduce a braking force applied to the articulable tip without entirely unlocking the articulable tip.

Some implementations may further comprise reducing a downward force applied to the articulation control piece to selectively increase a braking force applied to the articulable tip without fully locking the articulable tip.

Some implementations may further comprise applying different magnitudes of downward force to the articulation control piece to produce different levels of braking on the articulable tip.

In some implementations, the articulation control piece may be configured to be operated both to deflect the articulable tip and operate a tip braking feature using a single thumb with the handle being held using a one-handed grip.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of a laparoscope comprising an articulable tip according to some embodiments;

FIG. 2 is a cutaway view of the articulation control mechanism within the handle of the laparoscope of FIG. 1;

FIG. 3 is a cross-sectional view of the articulation control mechanism within the handle;

FIG. 4 depicts the articulation cables and pulleys of the articulation control mechanism;

FIG. 5 depicts the articulation control piece being pivoted to the right;

FIG. 6 depicts the articulation control piece being pivoted forward;

FIG. 7 is a cross-sectional view of an articulation control piece and corresponding automatic braking system according to other embodiments;

FIG. 8 is a perspective view of a borescope having an alternative articulation control mechanism and braking system; and

FIG. 9 is a lower perspective view of the borescope of FIG. 8 to illustrate the functionality of the alternative braking system.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

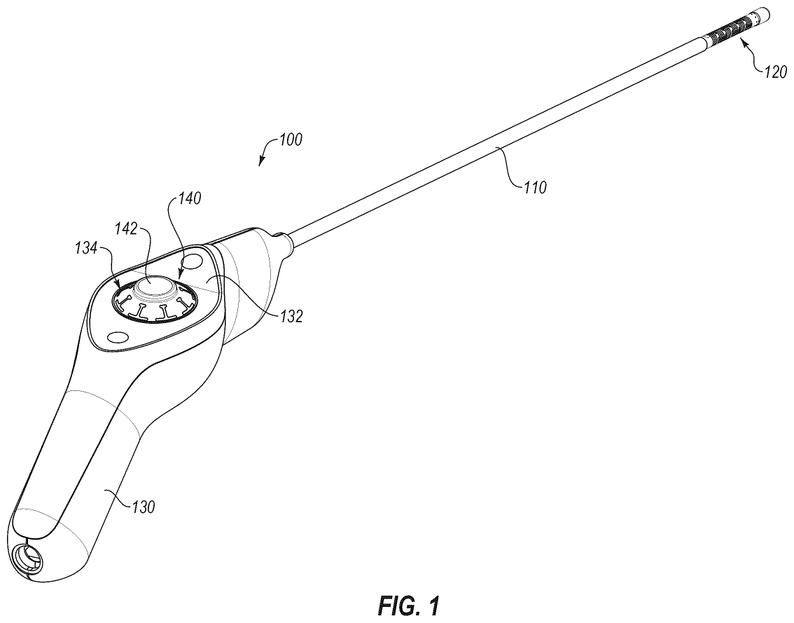

FIG. 1 depicts a laparoscope 100 according to some embodiments. Laparoscope 100 comprises a shaft 110 terminating at an articulable tip 120. Although not visible in FIG. 1, preferably tip 120 comprises an image sensor, a lens, one or more light sources, a microprocessor, power management chips, and/or a memory component.

A handle 130 is coupled with the shaft 110 at the proximal end of the shaft 110. Handle 130 comprises an upper surface 132, which may have a series of operating elements extending from or otherwise coupled thereto.

For example, an articulation control piece 140 may partially protrude from an opening 134 formed in the upper surface 132 of handle 130. Articulation control piece 140, which may also be referred to herein as a control “ball,” may comprise a spherical, hemispherical, or at least substantially spherical or hemispherical piece in some embodiments. In the depicted embodiment, articulation control piece 140 comprises a flattened upper surface 142, which may be configured to allow a surgeon/user to control the articulable tip 120 with a thumb.

In use, the articulation control piece 140 may be operated by first depressing the articulation control piece 140, which, as discussed in greater detail below, causes a locking mechanism associated with the articulation control piece 140 to unlock. After the locking mechanism has been released, the articulation control piece 140 can be pivoted and/or rotated in a desired direction. As discussed below, in preferred embodiments, the locking mechanism is configured to automatically re-lock and/or re-seat the articulation control piece 140—and thereby lock the articulable tip 120 in its current orientation—by simply releasing the articulation control piece 140.

In preferred embodiments, and as also discussed in greater detail below, the movement of the articulation control piece 140 in a particular direction results in articulation of the tip 120 in a corresponding direction that may be configured by linking certain bendable portions of the tip 120 to certain portions and/or sides of the articulation control piece 140 or an element coupled therewith. For example, pivoting or other movement of the articulation control piece 140 towards the left causes the tip 120 to bend to the left. Similarly, pivoting or other movement of the articulation control piece 140 to the right causes the tip 120 to bend to the right.

Bending of the tip 120 upwards and downwards may similarly be controlled by corresponding pivoting and/or rotation of the articulation control piece 140. In preferred embodiments, pivoting of the articulation control piece 140 forward may result in bending of the tip upwards. Similarly, pivoting of the articulation control piece 140 backwards may result in bending of the tip downwards. However, it should be noted that these directions may be reversed in some embodiments, depending upon the preferences of the user/surgeon. In other words, pivoting or other movement of the articulation control piece 140 forward may result in bending of the tip downwards and pivoting or other movement of the articulation control piece 140 backwards may result in bending of the tip upwards for users who find this configuration more intuitive.

In some embodiments, the articulation control piece 140 may be pivoted and/or rotated in any desired direction within a spherical workspace or portion of a spherical workspace. However, it is contemplated that other embodiments may be configured to limit pivoting/movement of the articulation control piece 140 to within only predetermined directions, such as only within two perpendicular directions and/or planes. For example, some embodiments may be configured to only allow a user to rotate/pivot the articulation control piece 140 side to side—which bends the tip 120 from side to side—and forward/backward—which bends the tip 120 up/down.

In some preferred embodiments, the laparoscope 100 or other borescope may comprise other elements not specifically discussed herein. For example, the tip 120 may comprise one or more light sources, such as LED lights, one or more image sensors, a lens assembly, and/or other medical borescope components. In some embodiments, the tip may further comprise a PCB and/or a memory element, such as a flash memory component or other non-volatile memory component, which may be used to store various types of data, such as the duration and/or number of uses of the device and/or model identification or calibration data, as described in U.S. patent application Ser. No. 14/958,728 titled MEDICAL BORESCOPES AND RELATED METHODS AND SYSTEMS, which was filed on Dec. 3, 2015 and is hereby incorporated herein by reference in its entirety.

As also described in the aforementioned patent application incorporated herein by reference, some embodiments may further comprise a reusable element, such as a dongle and/or pluggable box, which may be communicatively coupled with the scope, such as by way of wires or by being plugged into the scope. This dongle/box may comprise a memory element and/or a processor, which may be used to process image data from an image sensor in the device. In some embodiments, the dongle/box may be removably coupled with the scope so that it can be coupled with a plurality of distinct laparoscopes or other borescopes. For example, the dongle/box may comprise a data port that may be used to couple the dongle/box with a plurality of distinct borescopes and/or other devices, such as a general-purpose computer. In this manner, as discussed above, data obtained from the borescope, such as usage data, may be stored in the memory element of the dongle/box and ultimately transferred to another computer/device following a medical procedure.

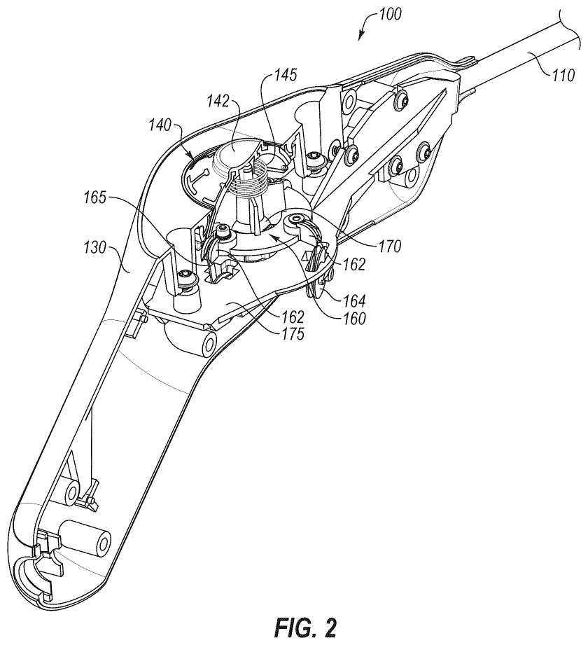

FIG. 2 is a cut-away view showing the internal components associated with the articulation control piece 140 within the handle 130. For example, FIG. 2 shows a spring 145 operably coupled with articulation control piece 140 and configured to bias the articulation control piece 140 towards a locking mechanism, which will be discussed in greater detail below. Spring 145, or another biasing member and/or mechanism, may be used to seat the articulation control piece 140 in a locking configuration such that a user can release the mechanism by depressing the articulation control piece 140. Depression of the articulation control piece 140 may be facilitated by flattened surface 142.

Upon depressing the articulation control piece 140, as mentioned above, the articulation control piece 140 may then be pivoted and/or rotated in a desired direction to cause corresponding bending/articulation of tip 120. After the tip 120 has been articulated to a desired configuration, the user may release the depressing force upon articulation control piece 140, which causes the articulation control piece 140 to re-seat and/or engage a locking mechanism (discussed further below). This re-seating/locking may allow the user to temporarily lock the tip 120 in place within the desired configuration without requiring any further control/force.

In some embodiments, the unseating/unlocking may be configured to be non-binary. In some such cases, the level of braking applied may be controllable. For example, the user may, in some cases, apply a selected amount of downward force on the articulation control piece corresponding with a desired level of braking. In preferred embodiments, this may result in increased force upon the articulation control piece 140 resulting in decreased braking force, and vice versa. This modulation of the braking force allows the user to adjust the sensitivity of the control mechanism, which in some cases may allow for directly affecting the sensitivity of the steerable portion of the scope 100 as well. In other words, by depressing the articulation control piece 140 lightly, or not at all, the steerable portion of the scope 100 may be more rigidly established in its current configuration compared to a configuration in which the user is using more force upon the articulation control piece 140, thereby resulting in greater ease of movement and/or flexibility of the steerable portion of the scope 100.

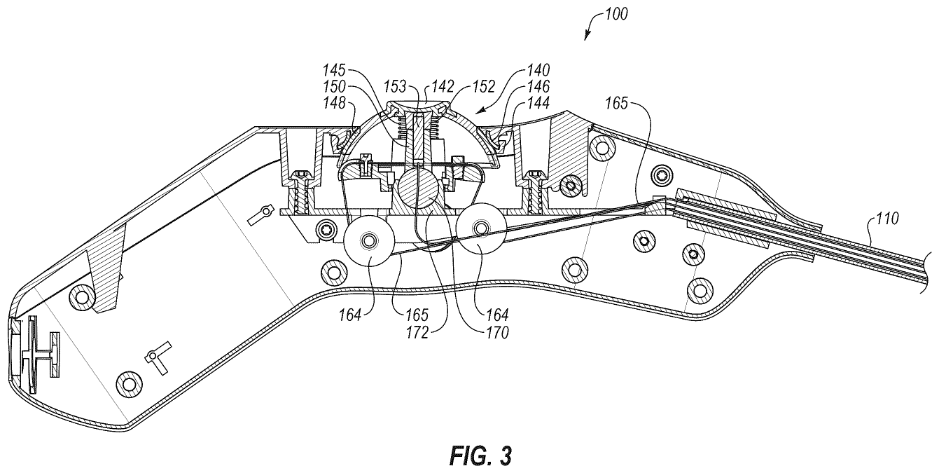

As shown in FIG. 2 and the cross-sectional view of FIG. 3, the articulation control piece 140 may be coupled with a swashplate 160. Swashplate 160 may have a central axis defined by a hollow cylinder 150, which may have a rod 153 positioned therein. An upper portion of rod 153 may be configured to protrude above the upper surface of hollow cylinder 150, which upper portion may be configured to be seated within a pin retention pocket 152 that extends downward from articulation control piece 140, in some cases as an integral portion thereof. Pin retention pocket 152 may be configured to be movable relative to hollow cylinder 150 of swashplate 160 to accommodate the movement provided by spring 145. The lower portion of cylinder 150 may be configured to receive and/or couple each of a plurality of articulation cables 165, which are discussed below.

The lower portion of cylinder 150 may be seated on a ball bearing 170 to allow for rotation and/or pivoting of the swashplate 160 and/or the articulation control piece 140. Ball bearing 170 may, in turn, be seated within a hemispherical seat 172 formed within a pulley mount 175. Hemispherical seat 172 may be molded around ball bearing 170, preferably with a material having a low coefficient of friction. Spring 145 may be seated on a plurality of spokes extending from cylinder 150.

In an alternative embodiment, this hemispherical seat 172 may have a plurality of lobes or teeth extending toward the swashplate 160, and the corresponding swashplate 160 may have a corresponding number of gaps within which these teeth may fit to prevent rotation of the two objects in a z-axis relative each other, preferably without hindering pivoting about the x and y axes.

A plurality of pulleys 164 or, alternatively, a plurality of cable troughs or the like, may be positioned within corresponding slots formed in pulley mount 175. A corresponding plurality of cable troughs 162 may be coupled with swashplate 160. Each of the cable troughs 162 preferably leads directly to a corresponding pulley 164 or corresponding secondary pulley trough positioned therebelow. However, a particular amount of space may be needed between each cable trough 162 and its corresponding pulley 164 to allow for pivoting/movement of swashplate 160.

As discussed below in greater detail, each pulley 164 may receive a specific articulation cable 165, each of which may be configured to bend tip 120 in a particular direction. In the depicted embodiment, as discussed above, there are four basic directions for articulation of tip 120. Thus, there are four corresponding articulation cables 165 and four associated pulleys 164. Each of the articulation cables 165 extends down a hollow portion or tube of shaft 110 to ultimately couple with a particular portion of the tip 120 such that, upon pulling of a particular articulation cable 165, the tip 120 will bend in the desired direction. In some embodiments, the cables 165 may be coupled with tip 120 via, for example, laser welding, adhesive, or other means.

In some cases, a mixing of the four basic directions may be provided. In other words, the tip may be articulated in various directions in between the four basic directions, thereby resulting in a finer control of the movement of the tip 120. For example, a control input of up and left may be configured to result in a corresponding tip movement of up and left, and so forth.

As best seen in the cross-sectional view of FIG. 3, in some embodiments, a braking mechanism may be provided for locking the position of articulation control piece 140, and thereby locking the orientation of articulable tip 120, in any desired position/configuration. As previously mentioned, a spring 145 or other resilient and/or biasing member may be used as part of this locking mechanism. Upon release of spring 145 or another biasing member, the force of spring 145 may cause articulation control piece 140 to re-seat, lock, and/or brake.

In the embodiment of FIG. 3, this braking and/or locking mechanism may include one or more structures configured to increase the friction between the articulation control piece 140 and the fixed structure of the handle 130, such as an inner portion of the upper surface 132 of handle 130 defining opening 134.

To accomplish this increase in friction to lock the articulation control piece 140 in place, the embodiment of FIG. 3 includes an embedded braking structure 146, which may be held in place using a clip 144, which may seat one end of the braking structure 146. The opposite end may be embedded within the structure of handle 130. Braking structure 146 may be made of a material with higher coefficient of friction than the surrounding material of the handle 130, such as rubber, a thermoplastic, such as thermoplastic polyurethane (TPU), or a rubber blend, such as Santoprene.

In some embodiments, the braking structure 146 may comprise a ring. In some such embodiments, the ring 146 may comprise a protruding and/or convex outer surface that is configured to contact a corresponding outer surface of articulation control piece 140, as shown in FIG. 3. Such embodiments may be configured to distort the convex outer surface upon braking to increase the surface area, and thereby increase the frictional braking force of the ring 146. However, it should also be understood that a variety of alternative configurations are contemplated, some of which are disclosed in other embodiments herein and others of which would be apparent to those of ordinary skill in the art after having received the benefit of this disclosure. For example, in some embodiments, rather than a ring, one or more discrete, but non-circular elements may be provided for this purpose. As another example, in some embodiments, an O-ring may be used as the braking structure 146.

In the embodiment of FIG. 3, another braking feature may be provided on the outer surface of articulation control piece 140. For example, one or more frictional inserts 148 are shown in FIG. 3 on the outer surface of articulation control piece 140. In some embodiments, insert 148 may comprise an annular structure extending about a full perimeter of the articulation control piece 140. Alternatively, insert 148 may comprise one or more (in some cases, a plurality) of discrete and/or spaced pieces extending about this perimeter. In another embodiment, one or more portions of the outer surface of the articulation control piece 140, such as the majority or entirety of this outer surface in some cases, may be formed to serve as the braking feature. Insert 148, like braking structure 146, may comprise any suitable material having a higher coefficient of friction than the surrounding material, such as, for example, rubber, a thermoplastic, or a thermoplastic and/or rubber blend.

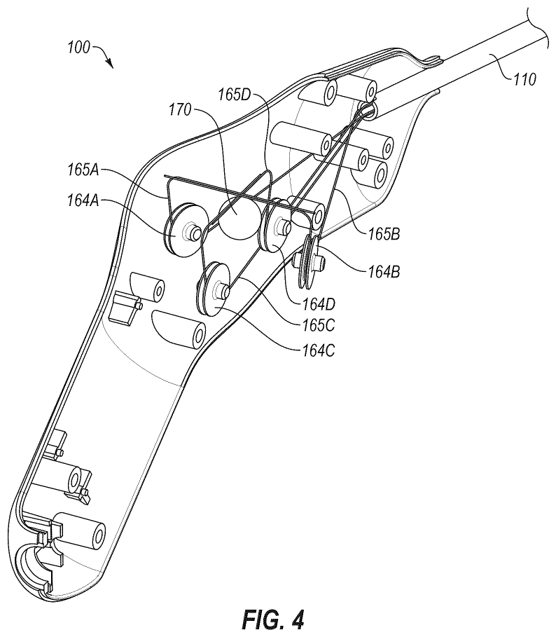

FIG. 4 depicts some of the key internal components associated with the articulation cables 165 with some of the previously discussed elements removed. This figure depicts a particular embodiment using pulleys, wherein each of the four pulleys 164A-164D and each of the corresponding articulation cables 165A-165D are represented. Each of the articulation cables 165 extends down a tubular portion of shaft 110 and ultimately couples with an opposing side of a bending section of tip 120 (not shown in this figure). As previously mentioned, however, in some alternative embodiments, these pulleys may be replaced with alternative means for guiding the articulation cables 165, such as slotted grooves or other static guiding elements.

Thus, articulation cable 165A extends through pulley 164A, which is positioned on the left side of the handle 130 of laparoscope 100, extends and ultimately couples with the right side of a bending section of tip 120. Similarly, articulation cable 165B extends through pulley 164B, which is positioned on the right side of the laparoscope 100, extends and ultimately couples with the left side of a bending section of tip 120.

Articulation cable 165C extends through pulley 164C, which is positioned on the proximal or rearward side of the handle 130 of laparoscope 100, extends and ultimately couples with the upper or top side of a bending section of tip 120. Similarly, articulation cable 165D extends through pulley 164D, which is positioned on the distal or forward side of the handle 130 of laparoscope 100, extends and ultimately couples with the lower or bottom side of a bending section of tip 120.

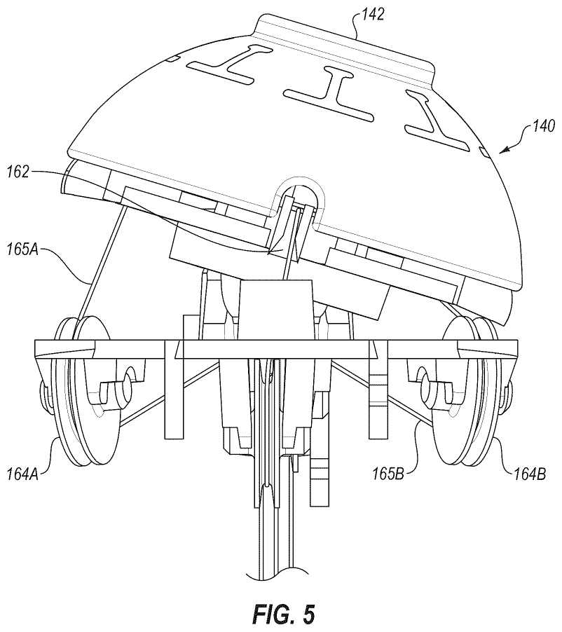

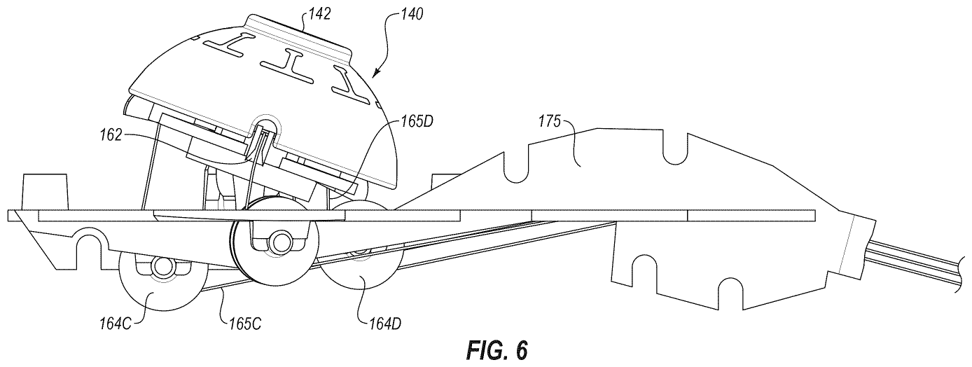

Further details regarding this articulation control scheme can be seen in FIGS. 5 and 6. FIG. 5 depicts articulation control piece 140 from behind and having been pivoted to the right. This pivoting motion causes the left articulation cable 165A to be pulled. Because the left articulation cable 165A is coupled with the right side of a bendable portion/section of the articulable tip 120, this movement results in articulation of the tip to the right. Thus, movement of the articulation control piece 140 to the right results in the intuitive movement of the tip 120 to the right as well.

Similarly, the movement of the articulation control piece 140 to the right also results in the relaxation of the right articulation cable 165B. Because of the coupling of the right articulation cable 165B with the left side of a bendable portion/section of the articulable tip 120, this relaxation further facilitates the desired bending of tip 120 to the right by allowing this bending to occur via the force delivered by the aforementioned tension on the left articulation cable 165A.

Of course, the opposite is true as well, and need not be shown in an additional figure to be demonstrated. That is, a pivoting motion of the articulation control piece 140 to the left causes the right articulation cable 165B to be pulled, which results in articulation of the tip to the left. Similarly, the movement of the articulation control piece 140 to the left also results in the relaxation of the left articulation cable 165A. Because of the coupling of the left articulation cable 165A with the right side of a bendable portion/section of the articulable tip 120, this relaxation further facilitates the desired bending of tip 120 to the left.

FIG. 6 is a side view of selected components of the articulation control mechanism during another pivoting motion. In this figure, the articulation control piece 140 has been pivoted distally or to the front of the laparoscope. This pivoting motion causes the rear articulation cable 165C to be tensioned or pulled. Because the rear articulation cable 165C is coupled with the top side/portion of the articulable tip 120, this movement results in articulation of the tip upwards. Thus, movement of the articulation control piece 140 in a forward, distal, and/or upward direction results in movement of the tip 120 upwards.

Similarly, the movement of the articulation control piece 140 in a forward, distal, and/or upward direction also results in the relaxation of the front/forward articulation cable 165D. Because of the coupling of the front articulation cable 165D with the bottom side and/or surface of a bendable portion/section of the articulable tip 120, this relaxation further facilitates the desired bending of tip 120 to upward by allowing this bending to occur via the force delivered by the aforementioned tension on the rear articulation cable 165C.

Again, the opposite is true as well. That is, a pivoting motion of the articulation control piece 140 in a rearward, proximal, and/or downward direction causes the front/forward articulation cable 165D to be pulled, which results in articulation of the tip in a downward direction. Similarly, the movement of the articulation control piece 140 in a rearward, proximal, and/or downward direction also results in the relaxation of the rear articulation cable 165C, which further facilitates the desired bending of tip 120 in a downward direction.

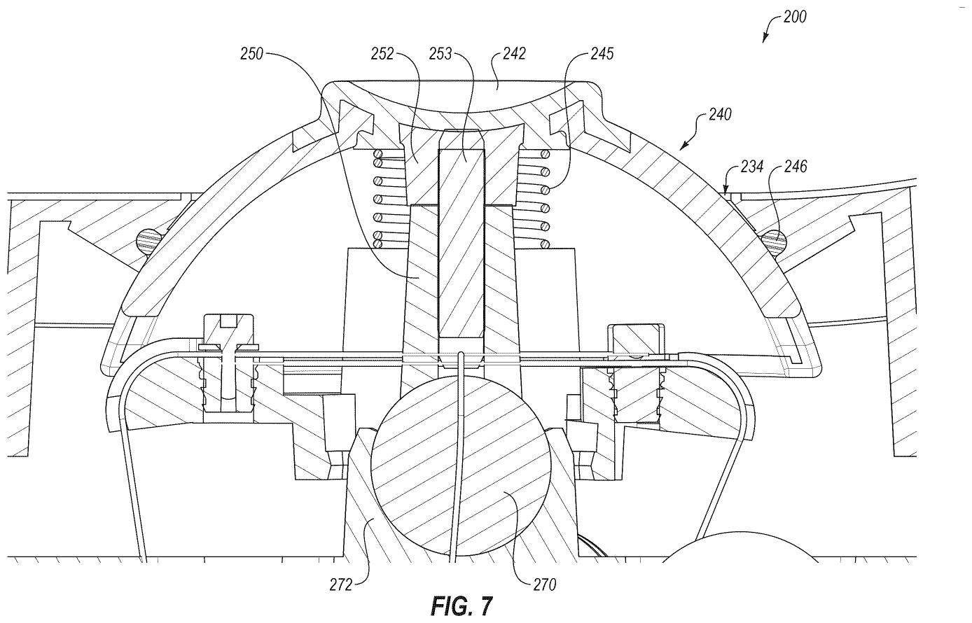

FIG. 7 is a close-up, cross-sectional view of an alternative embodiment of selected components of an articulation control mechanism of another laparoscope 200. This mechanism is similar to that of laparoscope 100 in many respects, but provides a distinct braking mechanism, as described in detail below. For example, an articulation control piece 240 partially protrudes from an opening 234 formed in the upper surface of the handle of laparoscope 200. Articulation control piece 240 may, again, comprise a spherical, hemispherical, or at least substantially spherical or hemispherical piece and may further comprise a flattened upper surface 242, which may be configured to allow a surgeon/user to control an articulable tip (not shown in FIG. 7) with her thumb. This flattened upper surface 242 may be slightly concave to provide a seat for the thumb, as shown in FIG. 7.

FIG. 7 further depicts a spring 245 operably coupled with articulation control piece 240 and configured to bias the articulation control piece 240 towards a locked/seated position and/or a locking mechanism. Spring 245, or another biasing member and/or mechanism, may be used to seat the articulation control piece 240 in a locking configuration such that a user can release the mechanism by depressing the articulation control piece 240.

Upon depressing the articulation control piece 240, as mentioned above, the articulation control piece 240 may then be pivoted and/or rotated in a desired direction to cause corresponding bending/articulation of the articulable tip. After the tip has been articulated to a desired configuration, the user may release the depressing force upon articulation control piece 240, which causes the articulation control piece 240 to re-seat and/or engage the aforementioned locking mechanism. This re-seating/locking may allow the user to temporarily lock the tip in place within the desired configuration without requiring any further control/force.

The locking/braking mechanism of laparoscope 200 differs from that of laparoscope 100 in that it comprises an O-ring 246 that is embedded in an inner portion of the structure of the handle adjacent to opening 234. O-ring 246 preferably protrudes from the slot within which it is embedded so as to allow for engagement with the exterior surface of articulation control piece 240 to provide enhanced friction and thereby prevent, or at least inhibit, the articulation control piece 240 from sliding/moving against the O-ring 246 in any configuration, including the neutral configuration depicted in FIG. 7, along with preferably any pivoted/articulated configuration, which may allow a surgeon to maintain the tip of the laparoscope 200 in a desired orientation without requiring force/attention to maintaining this orientation during a procedure.

The articulation control piece 240 may be coupled with a swashplate or other suitable structure to allow for transferring a pivoting and/or rotational force to a bending force on a tip of the scope 200, as previously mentioned. In the depicted embodiment, this structure has a central axis defined by a hollow cylinder 250, which may have a rod 253 positioned therein. Hollow cylinder 250 may be moveably coupled to a pin retention pocket 252 that is configured to receive an upper portion of rod 253 therein. Pin retention pocket 252 extends from and is coupled with the articulation control piece 240. The pin retention pocket 252 may be fixedly coupled with the articulation control piece 240 or, alternatively, may extend from and be an integral portion of the articulation control piece 240. The pin retention pocket 252 may be movable relative to the hollow cylinder 250 to accommodate the movement provided by spring 245 and thereby provide the tension/force to seat/lock/brake the articulation control piece 240 in any desired configuration.

The lower portion of cylinder 250 may be seated on a ball bearing 270, as discussed above in connection with laparoscope 100, to allow for rotation and/or pivoting of the articulation control piece 240 and its accompanying components. Ball bearing 270 may, in turn, be seated within a hemispherical seat 272, which may be part of a frame and/or pulley mount structure within the handle.

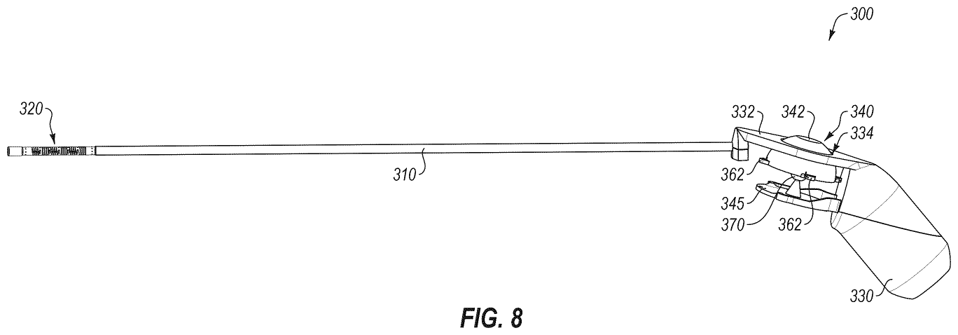

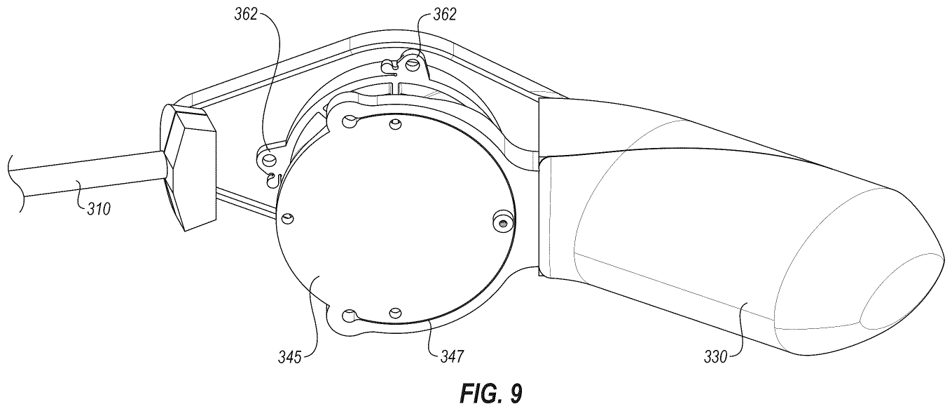

FIGS. 8 and 9 depict schematic views of still another laparoscope 300 according to other embodiments. Laparoscope 300 again comprises a shaft 310, through which various articulation cables, electrical wires, and/or optical elements may extend. An articulable tip 320 is positioned at the distal end of shaft 310. A handle 330 is positioned at the proximal end of shaft 310.

An alternative articulation control mechanism is used with the laparoscope 300 of FIGS. 8 and 9. As with previous embodiments, a hemispherical articulation control piece 340 is provided, which may comprise a flattened upper surface 342 to facilitate user engagement and pivoting as desired. Articulation control piece 340 is pivotably coupled with a smaller, spherical piece or bearing piece 370 positioned thereunder and fixed to a pedestal extending upward from a spring plate 345.

A portion of the articulation control piece 340 protrudes through an opening 334 formed along an upper surface 332 of the handle 330 to allow for access by a user. A user can articulate an articulable portion of the shaft, such as tip 320, by pressing down on the protruding portion of the articulation control piece 340 (typically with the thumb) and then rotating/pivoting the articulation control piece 340 on/about the spherical piece 370. Articulation cables can be coupled to the articulation control piece 340 via, for example, a series of eyelets 362 or other coupling features/points configured to transfer forces caused by pivoting of articulation control piece 340 to an articulable portion, such as an articulable tip 320, of the scope 300.

Although the upper surface of each of the articulation control pieces shown herein have flattened and/or slightly concave upper surfaces, in alternative embodiments, a “joystick” or other protruding control member may be used in connection with any of the embodiments disclosed herein or otherwise apparent to those of ordinary skill in the art after having received the benefit of this disclosure in order to provide further control.

Rotation/pivoting of the hemispherical control piece 340 in one direction causes the articulable tip 320 and distal tip to bend in a desired direction, as discussed above in connection with other embodiments.

As with other embodiments, laparoscope 300 further comprises an automatic braking system for the articulation control piece 340. The spherical/bearing piece 370 is coupled with a spring plate 345. When a user pushes on the protruding portion of the hemispherical control piece 340 to steer the articulating portion of the shaft/tip, the spring plate 345 is configured to resiliently move down as the user pivots/rotates the control piece 340. Upon release, the braking system—which is provided by a frictional engagement between the upper surface of the hemispherical control piece 340 and the portion of the handle defining opening 334—will automatically re-engage. This prevents the hemispherical control piece 340 from rotating, and thereby locks the shaft/tip in place at whatever angle/configuration it has been articulated to, upon release. Although not shown, any of the frictional engagement elements/features mentioned above may be used in this embodiment as well, if desired, such as an O-ring.

FIG. 9 illustrates the lower portion of the spring plate 345 to illustrate how the resilient force for operating the automatic braking function may operate. Spring plate 345 comprises a circular cut 347 that extends about a portion of its periphery, including a portion that may be coupled, in some cases integrally coupled, with handle 330. This allows the spring plate 345 to bend and move downward with the portion adjacent to circular cut 347 extending slightly below the adjacent portion thereof that extends into and couples with handle 330.

Again, the downward force provided by a user to unseat the articulation control piece 340 from its braking function and/or locked configuration causes the spring plate 345 to flex outward, thereby resulting in a restorative force upwards that also causes articulation control piece 340 to be forced upward. Upon release of this downward force, the material and/or shape of the spring plate 345 therefore provides a restorative force to re-seat the protruding portion of the articulation control piece 340, thereby automatically frictionally locking the articulation control piece 340 in its current position, and thereby locking the shaft/tip in its current configuration.

It will be understood by those having skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present invention should, therefore, be determined only by the following claims.

1. A medical borescope, comprising:
a handle comprising an opening;
a shaft extending from the handle;
a tip positioned at a distal end of the shaft; and
an articulation control mechanism, wherein the articulation control mechanism comprises:
an articulation control piece at least partially protruding from the opening, wherein the articulation control piece is operably coupled with the tip such that movement of the articulation control piece results in corresponding bending of the tip; and
an automatic braking system configured to automatically lock the tip in its current orientation upon releasing the articulation control piece.

2. The medical borescope of claim 1, wherein the articulation control piece comprises an at least partially spherical element.

3. The medical borescope of claim 1, wherein the articulation control piece is configured to unlock upon depressing the articulation control piece.

4. The medical borescope of claim 1, wherein the automatic braking system comprises a biasing element configured to bias the articulation control piece towards a locked configuration in which the tip is locked in place in its current angular orientation.

5. The medical borescope of claim 4, wherein the biasing element comprises a spring.

6. The medical borescope of claim 4, wherein the automatic braking system comprises a frictional braking surface configured to contact a corresponding braking surface of the articulation control piece.

7. The medical borescope of claim 6, wherein the automatic braking system further comprises a frictional insert formed on an outer surface of the articulation control piece, and wherein the frictional insert is configured to contact the frictional braking surface upon release of the articulation control piece.

8. The medical borescope of claim 6, wherein the frictional braking surface comprises a ring.

9. The medical borescope of claim 1, wherein the automatic braking system is configured to provide non-binary braking modulation.

10. A medical borescope, comprising:
a handle comprising an articulation control piece;
an articulable tip coupled with the handle, wherein the articulable tip is operably coupled with the articulation control piece to allow a user to pivot the articulable tip in at least four directions using corresponding movements of the articulation control piece, wherein the articulation control piece is configured to unlock upon depressing the articulation control piece and to automatically lock upon releasing the articulation control piece.

11. The medical borescope of claim 10, wherein the articulation control piece is biased towards a seated position in which a frictional interface frictionally locks an angular orientation of the articulable tip.

12. The medical borescope of claim 11, wherein the frictional interface is provided by a frictional insert formed along at least a portion of an outer surface of the articulation control piece.

13. The medical borescope of claim 11, wherein the frictional interface is provided by a frictional braking surface formed along a portion of an inner surface of the handle adjacent to an opening through which the articulation control piece at least partially extends.

14. The medical borescope of claim 10, wherein the articulation control piece comprises an at least partially spherical element.

15. The medical borescope of claim 10, wherein the articulation control piece is configured such that partial depression of the articulation control piece selectively reduces a braking force on the articulation control piece to allow for non-binary braking modulation.

16. A method for controlling articulation of a tip of a medical borescope, the method comprising the steps of:
depressing an articulation control piece to unlock an articulable tip of the medical borescope;
pivoting the articulation control piece in a selected direction to deflect the articulable tip in a corresponding direction; and
releasing the articulation control piece to automatically lock the articulable tip in its current angular orientation.

17. The method of claim 16, further comprising partially depressing the articulation control piece to selectively reduce a braking force applied to the articulable tip without entirely unlocking the articulable tip.

18. The method of claim 16, further comprising reducing a downward force applied to the articulation control piece to selectively increase a braking force applied to the articulable tip without fully locking the articulable tip.

19. The method of claim 16, wherein applying different magnitudes of downward force to the articulation control piece produces different levels of braking on the articulable tip.

20. The method of claim 16, wherein the articulation control piece is configured to be operated both to deflect the articulable tip and operate a tip braking feature using a single thumb with the handle being held using a one-handed grip.
